(19) United States
(12) Patent Application Publication
(10) Pub. No.: US 2014/0218685 A1
(43) Pub. Date: Aug. 7, 2014

(54) EYE REFRACTIVE POWER MEASURING APPARATUS

(71) Applicant: NIDEK CO., LTD., Gamagori-shi (JP)

(72) Inventor: Kenji NAKAMURA, Toyohashi-shi (JP)

(21) Appl. No.: 14/168,082

(22) Filed: Jan. 30, 2014

(30) Foreign Application Priority Data
Feb. 1, 2013 (JP) 2013-018599

(51) Int. Cl. A61B 3/00; A61B 3/103

(52) U.S. Cl. CPC A61B 3/0091; A61B 3/103
USPC 351/206; 351/221

(57) ABSTRACT

An eye refractive power measuring apparatus includes: a measuring part configured to project measurement light onto a fundus of an examinee's eye and measure refractive power of the eye based on reflection light of the measurement light from the fundus; a fixation target presenting part configured to present a fixation target to the eye; a drive part configured to move a presenting position of the fixation target; and a control part configured to control the drive part to move the presenting position from far distance to near distance, the apparatus being configured to measure the eye refractive power in at least a far position and a near position, wherein the control part controls the drive part to change a control amount thereof based on a change in measurement results of the eye refractive power while the fixation target is moved from the far distance to the near distance.

### CROSS-REFERENCE TO RELATED APPLICATIONS

This application is based upon and claims the benefit of priority from the prior Japanese Patent Application No. 2013-018599 filed on Feb. 1, 2013, the entire contents of which are incorporated herein by reference.

### BACKGROUND

The present disclosure relates to an eye refractive power measuring apparatus for measuring eye refractive power of an examinee's eye.

As an eye refractive power measuring apparatus for objectively measuring eye refractive power of an examinee's eye, there is known an eye refractive power measuring apparatus configured to change a presenting distance (a presenting position) of a fixation target at which the eye fixates to a plurality of presenting distances from a far point to a near point, obtain accommodation power (amplitude) of the eye based on measured refractive powers at the far point and the near point, and determine add power of the eye by use of the obtained accommodation power (see JP-A-2005-125086).

### SUMMARY

Meanwhile, when the refractive power for near distance (near vision) is to be measured using the conventional apparatus, the moving speed of a fixation target during movement from far to near is constant. Accordingly, when the presenting distance comes close to the near distance with respect to the examinee's eye, in some cases, the eye is unable to follow or track the fixation target and abandons tracking.

In a case of measuring the accommodation power of an examinee's eye, for instance, the accommodation power is calculated based on eye refractive power at the stage when the eye (the examinee) abandons tracking the fixation target. However, the position at which the eye abandons tracking does not always correspond to a limit position of the accommodation power of the eye. In some cases, accordingly, the actual accommodation power of the eye may be larger than that.

The present disclosure has been made to address the above problems and has a purpose to provide an eye refractive power measuring apparatus capable of well measuring refractive power of an examinee's eye for near distance.

To achieve the above purpose, an eye refractive power measuring apparatus provided as one typical embodiment is an eye refractive power measuring apparatus including: a measuring part configured to project measurement light onto a fundus of an examinee's eye and measure eye refractive power of the eye based on reflection light of the measurement light from the fundus; a fixation target presenting part configured to present a fixation target to the eye; a drive part configured to move a presenting position of the fixation target to be presented to the eye; and a control part configured to control the drive part to move the presenting position of the fixation target from far distance to near distance, the apparatus being configured to measure the eye refractive power in at least a far position and a near position, wherein the control part controls the drive part to change a control amount of the drive part based on a change in measurement results of the eye refractive power while the fixation target is moved from the far distance to the near distance.

### BRIEF DESCRIPTION OF THE DRAWINGS

FIG. 1 is an external view of an eye refractive power measuring apparatus in an embodiment of the present disclosure;

FIG. 2 is a schematic configuration view of optical systems and a control part;

FIGS. 3A and 3B are schematic diagrams to explain a configuration of a ring lens;

FIG. 4 is a diagram showing a ring image imaged by an imaging device;

FIG. 5 is a view showing an anterior segment image and various index images displayed on a monitor;

FIG. 6 is a first fixation target plate to be used in an accommodation measuring mode;

FIG. 7 is a flowchart to explain accommodation measurement;

FIG. 8 is a display example of the monitor during accommodation measurement;

FIG. 9 is a screen showing measured results of accommodation displayed on the monitor; and

FIG. 10 is a print example of measurement results including measured results of accommodation.

### DETAILED DESCRIPTION OF THE EXEMPLARY EMBODIMENTS

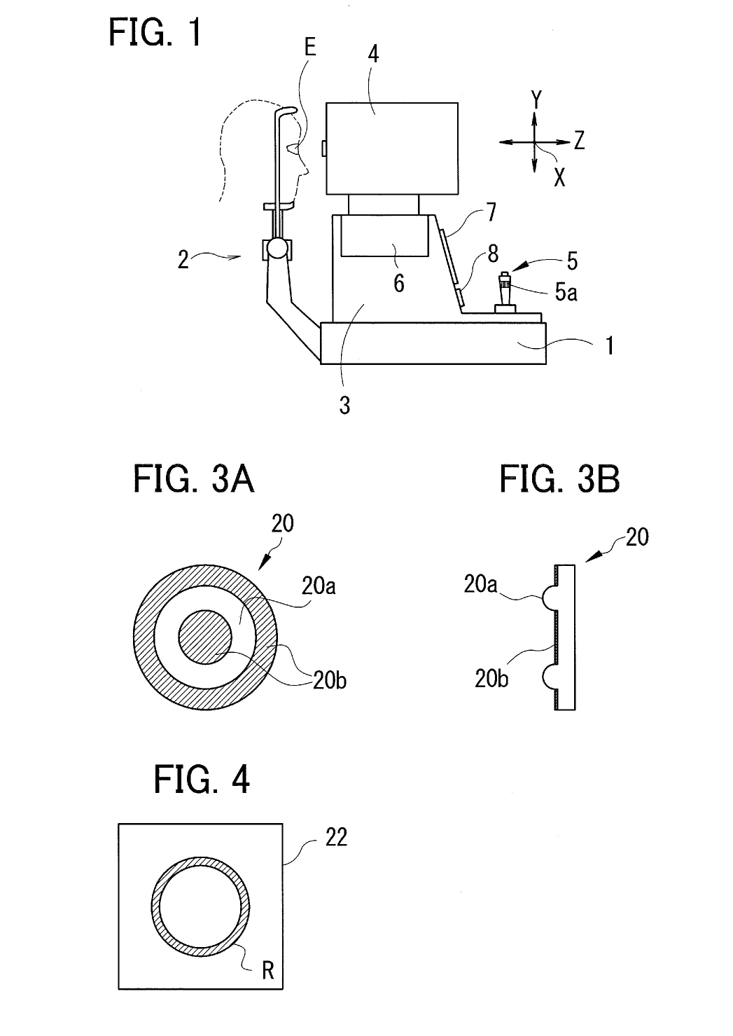

An eye refractive power measuring apparatus in an embodiment of this disclosure will be explained below referring to accompanying drawings. FIG. 1 is an external configuration view of the apparatus in the present embodiment. The measuring apparatus includes a base table 1, a face support unit 2 attached to the base table 1, a movable unit 3 provided movably on the base table 1, and a measuring part 4 provided movably on the movable unit 3 and configured to contain optical systems which will be mentioned later. The measuring part 4 is moved right and left (X direction), up and down (Y direction), and back and forth (Z direction) with respect to an examinee's eye E by an XYZ drive part 6 provided in the movable unit 3. The XYZ drive part 6 includes slide mechanisms provided one for each of the X, Y, and Z directions, motors, and others. The movable unit 3 is moved on the base table 1 in the X direction and the Z direction by operation of a joystick 5 and is moved in the Y direction by Y drive of the XYZ drive part 6 caused by rotation of a rotary knob 5a. The movable unit 3 is further provided with a monitor 7 for displaying various kinds of information such as an observation image and measurement results of the eye E, and a switch part 8 on which switches used for various settings are arranged.

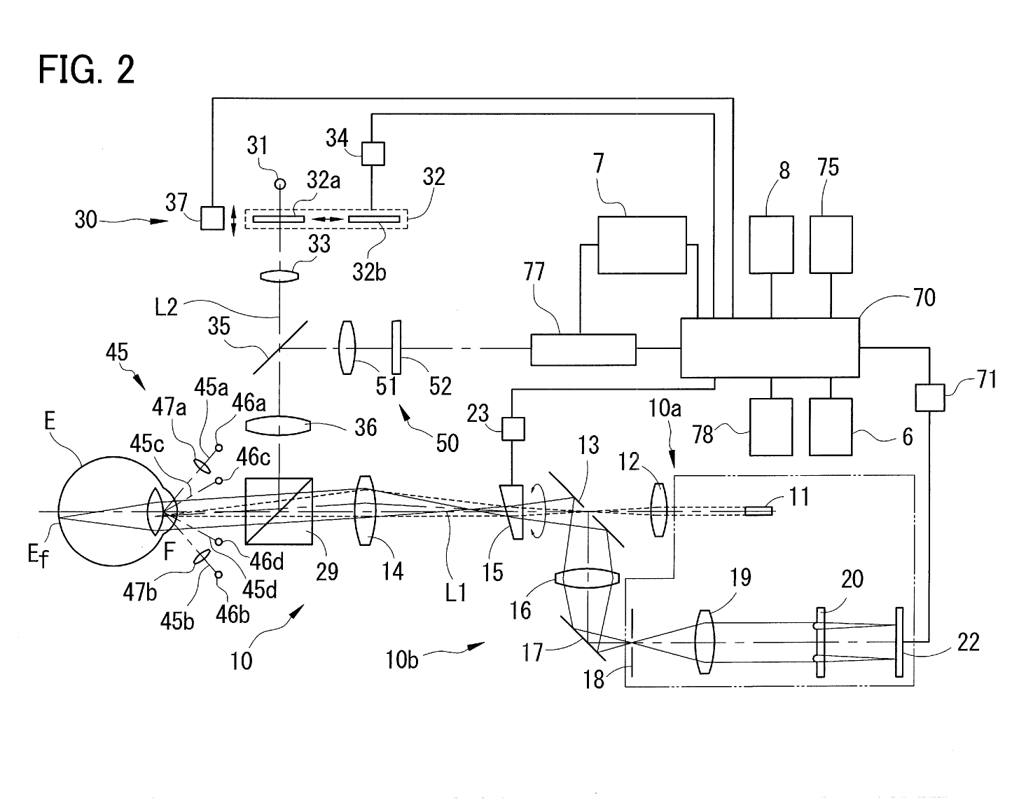

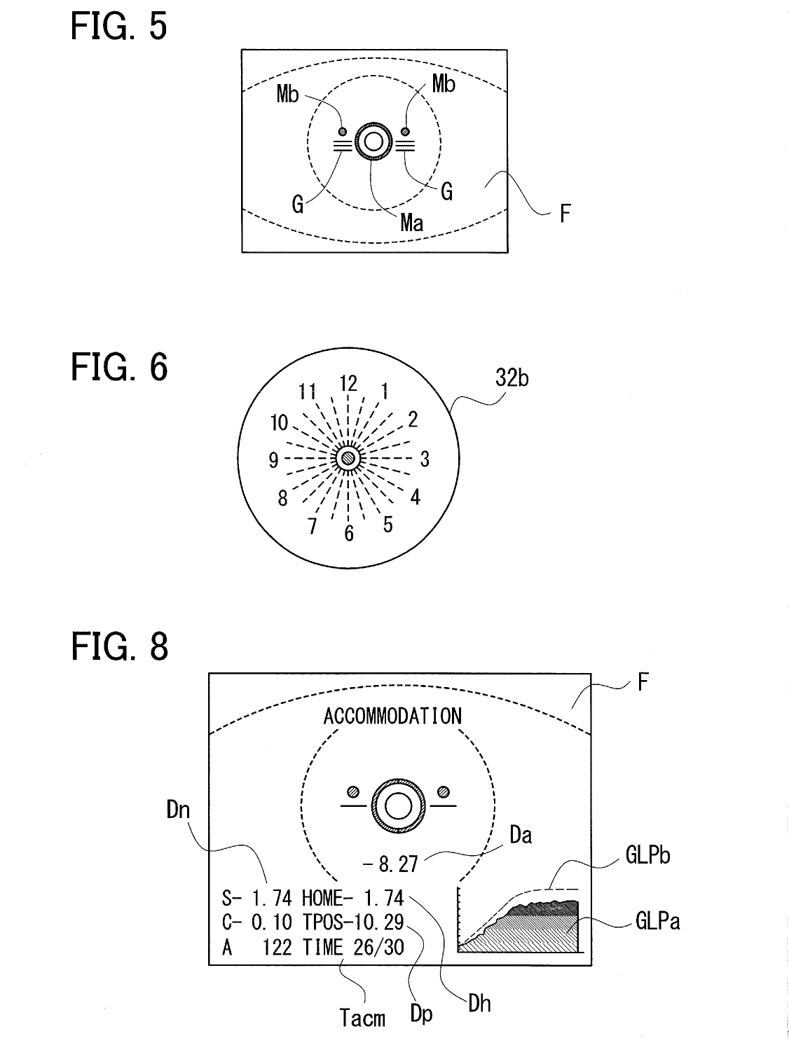

FIG. 2 is a schematic configuration view of optical systems and a control system of the present apparatus. A measuring optical system 10 includes a projecting optical system 10a for projecting spot-shaped measurement index light onto a fundus Ef of the eye E through the center of a pupil thereof and a light receiving optical system 10b for extracting the measurement index light reflected from the fundus Ef, in a ring form, through the periphery of the pupil.

The projecting optical system 10a includes, on an optical axis L1 of the measuring optical system 10, an infrared point light source 11 for measurement such as LED and SLD, a relay lens 12, a hole mirror 13, a prism 15 that is rotated about the optical axis L1 by a drive part 23, and an objective lens 14. This optical system 10a serves as light projecting means. The infrared point light source 11 is in an optically conjugate relationship with a fundus Ef of an emmetropic eye. An aperture of the hole mirror 13 is in an optically conjugate relationship with a pupil of the eye E. The term “conjugate” in the present specification means not only an exact conjugate relationship but also a conjugate relationship with accuracy required in relation to measurement accuracy.

The light receiving optical system 10b shares the objective lens 14, prism 15, and hole mirror 13 with the projecting optical system 10a, and further includes a relay lens 16 and a total reflection mirror 17 which are arranged on the optical axis L1 in a reflecting direction of the hole mirror 13, and a light receiving diaphragm 18, a collimator lens 19, a ring lens 20, and an imaging device 22 such as area CCD, which are arranged on the optical axis L1 in a reflecting direction of the total reflection mirror 17. The light receiving diaphragm 18 and the imaging device 22 are positioned in an optically conjugate relationship with the fundus Ef. As shown in FIGS. 3A and 3B, the ring lens 20 consists of a lens part 20a formed as a ring-shaped cylindrical lens on one side of a transparent flat plate and a shaded part 20b applied with coating for light shielding over an area other than the ring-shaped cylindrical lens part 20a. The ring lens 20 is in an optically conjugate relationship with the pupil of the eye E. Output from the imaging device 22 is input into a control part 70 via an image memory 71.

Between the objective lens 14 and the eye E, there is placed a beam splitter (a half mirror) 29 for delivering fixation target light from a fixation target presenting optical system 30 to the eye E and delivering reflection light from an anterior segment of the eye E to an observation optical system 50. In the present embodiment, the fixation target presenting optical system 30 is used as fixation target presenting means to present a fixation target to the eye E. The fixation target presenting optical system 30 includes for example a visible light source 31 for presenting fixation targets, a fixation target plate 32 having fixation targets, a light projecting lens 33, a half mirror 35, and an objective lens 36 for observation, which are arranged on an optical axis L2 to be made coaxial with the optical axis L1 by the beam splitter 29. The visible light source 31 and the fixation target plate 32 are moved along the optical axis L2 by a drive part 37 under control of the control part 70 to apply a fogging to the eye E. The fixation target plate 32 includes two kinds of fixation target plates; a first fixation target plate 32a to be used in objective measurement of far-vision (distant-vision) refractive power and a second fixation target plate 32b to be used in measurement of accommodation power of the eye E.

The first fixation target plate 32a and the second fixation target plate 32b can be switched from one to the other by the drive part 34 driven by the control part 70. In the present embodiment, the drive part 37 includes a stepping motor as an actuator and also uses in combination a photo interrupter serving as a reference position. The control part 70 that controls the drive part 37 with the stepping motor and the photo interrupter can detect the position of the fixation target plate 32 on the optical axis L2. Components constituting the drive part 37 are not limited to the above as long as the control part 70 can control to move the fixation target plate 32 and detect the position of the fixation target plate 32 on the optical axis L2. In the present embodiment, the drive part 37 is used as drive means to move a presenting position of a fixation target to be presented to the eye E. In the present embodiment, furthermore, the control part 70 is also used as control means to control the drive part 37 to move the presenting position of the fixation target from far distance (far vision) to near distance (near vision).

A Z-direction alignment index projecting optical system 45 is an optical system for projecting an alignment index for detection in the back and forth direction (Z direction) and includes two sets of first projecting optical systems 45a and 45b arranged symmetrically with respect to the optical axis L1, and two sets of second projecting optical systems 45c and 45d arranged symmetrically with respect to the optical axis L1 to provide optical axes arranged at a narrower angle than optical axes of the first projecting optical systems 45a and 45b. The first projecting optical systems 45a and 45b respectively include point light sources 46a and 46b that emit near infrared light and collimator lenses 47a and 47b to project infinite index images with almost parallel light onto the eye E. On the other hand, the second projecting optical systems 45c and 45d respectively include point light sources 46c and 46d that emit near infrared light to project finite index images with a divergent beam onto the eye E.

The observation optical system 50 shares the observation objective lens 36 and the half mirror 35 with the fixation target presenting optical system 30 and further includes an imaging lens 51 and an imaging device 52 arranged on an optical axis in a reflecting direction of the half mirror 35. The imaging device 52 is in an optically conjugate relationship with the anterior segment of the eye E. Output from the imaging device 52 is input into the control part 70 and the monitor 7 through an image processing part 77. An anterior segment image of the eye E formed by an anterior-segment illuminating light source not shown is imaged by the imaging device 52 and displayed as a moving image on the monitor 7. This observation optical system 50 is also used as an optical system for detecting an alignment index image (index images Ma and Mb which will be mentioned later) formed on a cornea of the eye E. The position of the alignment index image (the index images Ma and Mb) is detected by the image processing part 77 and the control part 70.

The control part 70 is connected to the image memory 71, a memory 75, the image processing part 77, the monitor 7, the XYZ drive part 6, the switch part 8, end others. The control part 70 controls the entire apparatus and also calculations refractive value and refractive power of the eye E, and others. In the present embodiment, the memory 75 is used as storage means.

When the refractive power of the eye E is to be determined, the control part 70 turns on the infrared point light source 11 for measurement upon receipt of a measurement start signal from the switch part 8 and also causes the drive part 23 to rotate the prism 15 at high speed. Measurement light emitted from the infrared point light source 11 is projected onto the fundus Ef through the components from the relay lens 12 to the beam splitter 29, thus forming a spot-shaped point-light-source image rotating on the fundus Ef. At that time, a pupil projection image (projection light on the pupil) of the aperture of the hole mirror 13 is eccentrically rotated at high speed by the prism 15 rotating about the optical axis L1. The prism 15 is rotated at a speed for two rotations per one light exposure time (light storage time) of the imaging device 22.

The light of the point-light-source image formed on the fundus Ef is reflected and scattered, and exits the eye E, and then is converged through the objective lens 14. This light is converged again on the aperture of the light receiving diaphragm 18 through the components from the high-speed rotating prism 15 to the total reflection mirror 17, made into almost parallel light (in a case of an emmetropic eye) by the collimator lens 19, and extracted as ring-shaped light by the ring lens 20. This light is received as a ring image by the imaging device 22.

The imaging devices 22 and 52 in the present embodiment are two-dimensional imaging devices, which employ a CCD (Charge Coupled Device) image sensor. The two-dimensional imaging devices may also employ a CMOS (Complementary Metal Oxide Semiconductor) image sensor. Furthermore, the imaging devices 22 and 52 in the present embodiment are operated synchronously in response to input/output signals. An imaging time interval of the imaging devices 22 and 52 is 1/30 seconds and one light exposure time thereof is also 1/30 seconds.

Operations of the apparatus configured as above will be explained below. The eye refractive power measuring apparatus in the present embodiment includes an objective far-vision refractive-power measuring mode to measure normal far-vision refractive power and an accommodation measuring mode to measure accommodation power of the eye E. An explanation is given first to the objective far-vision refractive-power measuring mode and then to the accommodation measuring mode. The objective far-vision refractive-power measuring mode is a measurement mode in which a fixation target is placed for far distance to precisely determine the eye refractive power of the eye E. The accommodation measuring mode is a measuring mode in which the presenting distance of a fixation target is changed to detect a far point and a near point, thereby determining accommodation power (amplitude) of the eye E.

An examiner asks an examinee to put his/her face on the face support unit 2 and then makes positional alignment of the measuring part 4 with the examinee's eye E by projecting an alignment index on the cornea of the eye E. Prior to this alignment with the eye E, the examiner operates the switch part 8 to select the objective far-vision refractive-power measuring mode in advance. The control part 70 detects an alignment state with respect to the eye E based on an imaging signal from the imaging device 52. The control part 70 calculates the center position (almost the corneal center) of an index image Ma to determine misalignments in the X and Y directions. The alignment state in the Z direction is detected from a positional relationship among four index images formed by the Z-direction alignment index projecting optical system 45. Whether or not the Z-direction alignment state is appropriate is detected by comparison between an image spacing between two infinite index images formed by the first projecting optical systems 45a and 45b and an image spacing between two finite images formed by the second projecting optical systems 45c and 45d. In the case of projecting the infinite indexes, the image spacing hardly changes even if the Z-direction alignment state is changed. On the other hand, in the case of projecting the finite indexes, the image spacing changes according to changes in the Z-direction alignment state. By utilizing this characteristic, the Z-direction alignment state can be determined (see JP-A-6 (1994)-46999). The control part 70 increases/decreases the number of indicators G based on a detection result of the Z-direction alignment.

The control part 70 moves the measuring part 4 in the X and Y directions based on the index image formed by the light sources 46c and 46d and moves the measuring part 4 in the Z direction based on the four index images formed by the Z-direction alignment index projecting optical system 45. When the alignment state in each of the X, Y, and Z directions falls within a predetermined range, the control part 70 judges that the alignment is completed, and automatically generates a measurement start signal to execute measurement. In the case of manual measurement, the examiner operates the joystick 5 and others to terminate alignment and then presses a measurement start switch not shown to input a measurement start signal.

Upon receipt of a trigger signal, the control part 70 turns on the measurement infrared point light source 11 to project a measurement index onto the fundus Ef. The control part 70 then receives the reflection light through the imaging device 22 and detects an index image (a ring image R). At that time, preliminary measurement is first performed and, based on a result thereof, the visible light 31 and the fixation target plate 32 for presenting fixation targets are moved in the optical axis direction, thereby fogging the eye E. Thereafter, the eye E is subjected to main measurement.

FIG. 4 shows a ring image imaged by the imaging device 22 in the measurement executed in response to the measurement start signal. An output signal from the imaging device 22 is stored as image data (ring picture) in the image memory 71. In the main measurement in the present embodiment, the ring picture (ring image R) is continuously captured and subjected to addition-accumulation processing. Under the basic condition that the number of addition processing times is one or two, the ring image is continuously captured by the imaging device 22 and a plurality of image data is stored as image data for the addition processing in the image memory 71.

Thereafter, the control part 70 creates the added image data by using the plurality of images stored in the image memory 71. The control part 70 specifies (thins) the position of the ring image in each of meridian directions based on the image data. Specifically, the control part 70 specifies the position of the ring image by cutting a waveform of a luminance signal by a predetermined threshold and determining a midpoint of the waveform at the cut position, a peak of the waveform of the luminance signal, a center gravity of the luminance signal, etc. When noise light apt to be superimposed on the image data is suppressed by the addition processing, a measurement result can be obtained with accuracy (for the details, refer to JP-A-2006-187482).

Next, the control part 70 approximates the ring image to an elliptic shape by a least-square method or the like based on the specified image position of the ring image. This ellipse approximation method can use an ellipse approximation formula well known in eye refractive power measurement, corneal shape measurement, and others. A refractive error in each of the meridian directions can be determined from the approximated elliptic shape. Based on this result, accordingly, the refractive power of the examinee's eye, i.e., S (Sphere power), C (Cylinder power), and A (Astigmatic axial angle), is calculated. These measurement results are displayed on the monitor 7.

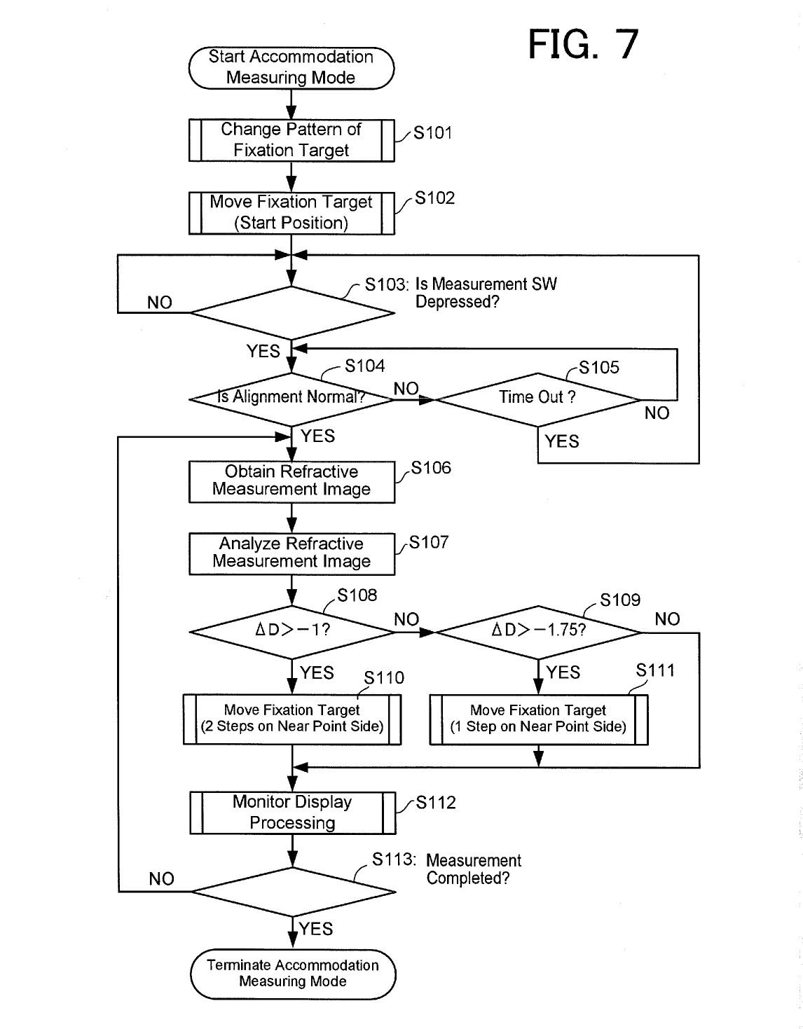

A flow of the accommodation measuring mode is next explained referring to FIG. 7. The control part 70 detects based on the input signal from the switch part 8 that the examiner has changed the measurement mode from the objective far-vision refractive-power measuring mode to the accommodation measuring mode. In the accommodation measuring mode, as in the objective far-vision refractive-power measuring mode, an anterior segment image F (moving image) of the eye E is displayed on the monitor 7 based on the output signal of the imaging device 52. Furthermore, alignment detection and movement of the measuring part 4 in the X, Y, and Z directions are performed as in the objective far-vision refractive-power measuring mode.

In the flow in FIG. 7, the control part 70 controls the drive part 37 to change a control amount of the drive part 37 while moving the fixation target from the far distance to the near distance (see steps S108 to S111 in FIG. 7). The fixation target used in the present embodiment is the second fixation target plate 32b, but is not limited thereto, and may be the first fixation target plate 32a. To change the control amount, for example, the control part 70 changes at least one of the moving speed of a fixation target while the fixation target is moved from the far distance to the near distance and the moving amount of a fixation target at each step while the fixation target is moved on a step-by-step basis from the far distance to the near distance.

While the fixation target is moved from the far distance to the near distance, the control part 70 in the present embodiment monitors in real time the information of the presenting position of the fixation target and the measurement results of the eye refractive power. Those monitoring results are used to change the control amount. In monitoring, for example, the control part 70 obtains the presenting position information and the measurement results continuously or at predetermined time intervals, and updates them continually. The control part 70 may cause the memory 75 to store the measurement results obtained at each position in association with the presenting positions.

When the control amount is to be changed by use of the monitoring results, in the present embodiment, the control part 70 changes the control amount based on the presenting position information of the fixation target and the measurement results of the eye refractive power at the presenting distance as shown in FIG. 7. Thus, the tracking state of the eye E with respect to the fixation target is reflected in the control amount. For instance, the control part 70 may be configured to decrease the moving speed of the fixation target or the moving amount in each step if the presenting position of the fixation target exceeds an allowable range with respect to a refractive value. The control part 70 may also be configured to control movement of the fixation target so that the presenting position of the fixation target does not exceed the allowable range with respect to the eye refractive power during current measurement. The presenting position of the fixation target and the measurement results of the eye refractive power are used by conversion into for example a diopter value (D) or a distance value (m). The control part 70 may also be configured to change the control amount of the drive part 37 so that the presenting position of the fixation target does not differ by a set threshold or more from a measurement result on a most negative side measured while the fixation target is moved from the far distance to the near distance.

FIG. 8 is a display screen of the monitor 7 in the accommodation measuring mode. The monitor 7 displays thereon the anterior segment image F (moving image) of the eye E, measurement values (S, C, A) measured in the accommodation measuring mode, a refractive power minimum value Dh representing a minimum value of refractive power (diopter) during measurement of accommodation power, a fixation target corresponding value Dp obtained by converting the current presenting distance of the fixation target into a diopter, and a measurement elapsed time Tacm indicating an elapsed time counted from the start of accommodation measurement. As will be mentioned later, a last accommodation power Da obtained by subtracting the refractive power measured at the current presenting distance of the fixation target from the refractive power minimum value Dh corresponding to a far point, a refractive power change graph GLPa representing changes in the last accommodation power Da associated with changes in presenting distance of the fixation target, and a fixation target conversion graph GLPb representing the presenting distance of the moving fixation target plotted by conversion into diopter are displayed during measurement of accommodation power. In the refractive power change graph GLPa and the fixation target conversion graph GLPb, the lateral axis of indicates time (seconds) and the vertical axis indicates diopter (diopter value).

<Step S101>

The control part 70 controls the drive part 34 to change the type of the fixation target plate 32 from the first fixation target plate 32a used in the objective far-vision refractive-power measuring mode to the second fixation target plate 32b to be used in the accommodation measuring mode. FIG. 6 shows the second fixation target plate 32b to be used in the accommodation measuring mode. This second fixation target plate 32b includes geometric patterns consisting of circles and lines, and letters. Although the present embodiment uses the fixation target plates 32 having different patterns between the objective far-vision refractive-power measuring mode and the accommodation measuring mode, the present disclosure is not limited thereto. The same fixation target plate may be used in both of the objective far-vision refractive-power measuring mode and the accommodation measuring mode.

<Step S102>

The control part 70 controls the drive part 37 to move the fixation target to a position for far distance displaced by 0.5 diopter from the far-vision refractive power Dn measured in the objective far-vision refractive-power measuring mode. In the accommodation measuring mode, the presenting distance of the fixation target is moved from far to near. Since the index is placed in a position for far distance more than the far-vision refractive power Dn of the eye E accurately measured in the objective far-vision refractive-power measuring mode, and the presenting distance of the fixation target passes the distance corresponding to the far-vision refractive power Dn of the eye E during measurement of the accommodation power from the far distance toward the near distance, it is possible to ensure tracking ability (visibility) to the fixation target at an initial stage in the accommodation measurement and detect the far point promptly.

<Step S103>

After the completion of changing the fixation target (the fixation target plate) in step S101 and moving the fixation target in step S102, the control part 70 monitors the status of a start switch not shown placed at the tip of the joystick 5. When the control part 70 detects that the start switch is depressed by the examiner to start the accommodation measurement (S103: YES), the control part 70 outputs the measurement start signal and advances to step S104. While the start switch remains unchanged (S103: NO), the control part 70 is in a standby state in step S103 to wait for next measurement. In the accommodation measurement, the eye E has to follow or track the fixation target presented at different presenting distances. Thus, it is preferable that, before start of the measurement in the accommodation measuring mode, the examiner asks the examinee to follow the fixation target presented at changing distances. In the present embodiment, differing from the objective far-vision refractive-power measuring mode, the control part 70 disables automatic start of accommodation measurement even when the alignment state of the measuring part 4 with the eye E falls within a predetermined allowable range, and causes the monitor 7 to display a message or pattern (icon) indicating the completion of preparation when changing the fixation target and changing the presenting distance of the presenting position of the fixation target are completed.

<Step S104>

When the control part 70 detects that the start switch is depressed (S103: YES), the control part 70 determines the alignment state of the measuring part 4 with the eye E (S 104). When the eye E and the measuring part 4 are in an alignment state falling within the predetermined allowable range, the flow advances to step S106. When they are not in the alignment state falling within the predetermined allowable range, the flow goes to step S105. The predetermined allowable range is the same as in the alignment detection condition in the objective far-vision refractive-power measuring mode.

<Step S105>

In step S105, an elapsed time for which the alignment state does not fall within the predetermined allowable range from the time when the alignment state of the measuring part 4 with the eye E is detected immediately after the start switch is depressed is compared with a predetermined judgment time. If the elapsed time does not reach the predetermined judgment time (S105: NO), the flow returns to step S104. If the elapsed time reaches the predetermined judgment time (S105: YES), the measurement is interrupted and the flow returns to step S 103. In the present embodiment, when the elapsed time for which the alignment state does not fall within the predetermined allowable range continues for 5 seconds or more immediately after the start switch is depressed, it is judged as an error and the measurement is stopped. When the measurement is interrupted and the flow returns to step S103, the monitor 7 is caused to display a pattern (icon) indicating that the measurement is started upon re-depression of the start switch.

<Step S106>

The control part 70 monitors a vertical synchronization signal output from the imaging device 22 and waits for the timing to cause the imaging device 22 to newly start the light exposure period. At the timing when the light exposure period is newly started, the control part 70 monitors the vertical synchronization signal output from the imaging device 22 and waits by the light exposure period needed to calculate the refractive power. Herein, a method of measuring refractive power to determine an initial presenting position of a fixation target in the objective far-vision refractive-power measuring mode and accommodation measurement (first refractive power measurement) and a method of measuring refractive power during accommodation measurement in the accommodation measuring mode (second refractive power measurement) are configured to be different from each other. In the refractive power measurement during accommodation measurement (second refractive power measurement), there is no need to apply a fogging to the eye E. In the accommodation measuring mode, furthermore, the light exposure period (time) of the imaging device 22 needed to calculate the refractive power of the eye E is set to a shorter time than a standby time required in the objective far-vision refractive-power measuring mode. In the objective far-vision refractive-power measuring mode, the addition processing is performed on the output signal of the imaging device 22 in order to measure the far-vision refractive power of the eye E with accuracy. On the other hand, in the accommodation measuring mode, the addition processing is not performed for the purpose of prompt measurement.

In more detail, in the objective far-vision refractive-power measuring mode, one or two additions are performed by using the output signal (one image) successively output at an interval of 1/30 seconds from the imaging device 22. Since two additions are performed by three images, the light exposure time needed to calculate the refractive power takes up to about 100 ms. On the other hand, in the accommodation measuring mode, no addition is performed and the refractive power is calculated by only one image, so that the light exposure time needed to calculate the refractive power is 1/30 seconds (about 33 ms).

The refractive power of the eye E varies according to the presenting distance of the fixation target. When the presenting distance of the fixation target is moved (changed) during a light exposure period of the imaging device 22, a plurality of components of refractive power generated by change of the presenting distance of the fixation target are superimposed on a fixation target image (a ring image) received by the imaging device 22, resulting in a decrease in accuracy of the refractive power. The accommodation power is measured by obtaining a far point and a near point based on changes in refractive power. Thus, in case the presenting distance of the fixation target is changed during light exposure period of the imaging device 22, the accommodation power is obtained based on a low-reliable refractive power. In the present embodiment, therefore, the accommodation power is measured by measuring the refractive power of the eye E while nearly continuously moving the presenting distance of the fixation target from far to near, but the control part 70 performs the control not to move the presenting distance of the fixation target during light exposure period of the imaging device 22 required to obtain the refractive power (i.e., the control to temporarily stop movement of the fixation target). Specifically, the controller 70 causes the imaging device 22 to be exposed to light and then determines a refractive power by use of an output signal from the imaging device 22 while the fixation target remains stationary or at rest.

<Step S 107>

The control part 70 calculates the refractive power of the eye E at the presenting distance of the relevant fixation target based on the output signal of the imaging device 22 obtained in step S106. This refractive power measurement is performed by the same method as in the objective far-vision refractive-power measuring mode excepting inexecution of the addition processing.

<Step S108>

The control part 70 determines the tracking state of the eye E with respect to the fixation target based on a displacement amount between the information of the presenting position of the fixation target and a measurement result of the eye refractive power.

In more detail, the control part 70 subtracts the refractive power (a diopter value) obtained in step S107 from the diopter value obtained based on the presenting distance of the fixation target to calculate a tracking evaluation value ΔD (a diopter value) of the eye E at the presenting distance of the relevant fixation target. In step S108, the tracking evaluation value ΔD and a predetermined condition (a first condition) are compared. If the tracking evaluation value ΔD is larger than −1 diopter (S108: YES), the flow goes to step S110. If the tracking evaluation value ΔD is equal to or lower than −1 diopter (S108: NO), the flow goes to step S109. In the present embodiment, when the eye E has good accommodation tracking ability with respect to the moved presenting distance of the fixation target, the tracking evaluation value ΔD assumes a larger value than −1 diopter (e.g., −0.5 diopter). As the eye E has lower tracking ability, the tracking evaluation value ΔD assumes a smaller value (e.g., −2.0 diopter).

<Step S109>

The control part 70 compares the tracking evaluation value ΔD with the predetermined condition in a similar manner to in step S 108, but under a second condition which is a different comparative condition from that in step S 108. If the tracking evaluation value ΔD is larger than −1.75 diopter (S109: YES), the flow advances to step S111. If the tracking evaluation value ΔD is equal to or lower than −1.75 diopter (S109: NO), the flow goes to step S112.

<Steps S110, S111>

The control part 70 changes the control amount of the fixation target based on the aforementioned determination result of the tracking state. The control part 70 further changes the movement control of the fixation target based on the information of the presenting position of the fixation target and the measurement results of the eye refractive power at that presenting distance.

In more detail, the control part 70 moves the presenting distance of the fixation target based on the results determined in steps S108 and S 109. In step S110, the fixation target is moved to the near distance by two steps from the current presenting distance of the fixation target. In step S111, the fixation target is moved to the near distance by one step. In the present embodiment, when the drive part 37 is controlled to move the presenting distance of the fixation target by one step, the presenting distance of the fixation target is moved to a distance apart by 0.05 diopter in terms of diopter. The control part 70 judges whether or not the fixation target is moving based on whether or not a predetermined time has elapsed from the time when controlling the drive part 37 is started. The manner of judging whether or not the fixation target is moving is not limited thereto. For this purpose, detection means for detecting a moving amount may be provided in a place to which the second fixation target plate 32b is moved.

As explained above, the control part 70 calculates the accommodation tracking state of the eye E from the diopter value based on the presenting distance of the fixation target and the refractive power of the eye E measured at the relevant presenting distance, and reflects it in the control of changing the presenting distance of the fixation target. In other words, when the presenting distance of the fixation target is changed from the far distance (near a far point) toward the near distance, the limit of accommodation power of the eye E gradually appears. This results in lowering of the tracking ability of the eye E with respect to the fixation target (increasing of a difference between a diopter value resulting from the presenting distance of the fixation target and a diopter value corresponding to the measured refractive power of the eye E).

The control part 70 detects the accommodation tracking state of the eye E from the presenting distance of the fixation target and the measured refractive power. The control part 70 controls movement of the fixation target based on the detected accommodation tracking state so that the examinee does not abandon accommodation earlier than the limit of the accommodation power of the eye E. If the eye E is in a good accommodation tracking state, for instance, the control part 70 largely (quickly) moves the presenting distance of the fixation target. If the accommodation tracking state of the eye E deteriorates (or when the eye E approaches the limit of accommodation power), the control part 70 controls to reduce movement of the presenting distance of the fixation target or wait the accommodation tracking of the eye E. Accordingly, it is possible to measure the accommodation power of the eye E for a short required time while ensuring the accommodation tracking ability of the eye E.

In the present embodiment, if the tracking evaluation value ΔD is equal to or less than −1.75 (second condition) in step S109, the flow goes to step S112 and the presenting position of the fixation target remains stopped. Herein, there may be provided a third condition to further determine the tracking evaluation value ΔD in a section between step S109 in which the comparative result is determined as NO and step S112 following step S109. As the third condition, for example, if the tracking evaluation value ΔD is smaller than −2 diopter, the presenting position of the fixation target is moved for far distance by one step. If the tracking evaluation value ΔD is equal to or larger than −2 diopter, the flow goes to step S112. When the detected accommodation tracking state shows that the eye E clearly has low accommodation tracking, the control part 70 may return the presenting distance of the fixation target in an opposite direction to the moving direction of the presenting distance of the fixation target and perform the control to help or promote the accommodation tracking of the eye E.

In the present embodiment, when the presenting distance of the fixation target is to be changed based on the accommodation tracking state of the eye E, the control part 70 changes only the moving distance of the fixation target at a constant speed. Since the accommodation tracking ability of the eye E is changed even by the moving speed of the fixation target, the control part 70 may be configured to change the moving speed of the presenting distance of the fixation target based on the detected accommodation tracking state of the eye E. In the present embodiment, one cycle (S106-S113) during accommodation measurement is about 83 ms. About 40% of the one cycle corresponds to a light receiving period of the imaging device 22. Further, the aforementioned one cycle during accommodation measurement is continuously performed up to completion of measurement. In the present embodiment, the control part 70 controls to change only the moving distance of the fixation target without changing the moving speed. However, in terms of one cycle, the control of changing the moving distance and the control of changing the moving speed are not so different.

<Step S 112>

The control part 70 displays on the monitor 7 the measured last accommodation power Da and plots the refractive power change graph GLPa and the fixation target conversion graph GLPb. The last accommodation power Da becomes a value (diopter) obtained by subtracting the fixation target corresponding value Dp from the refractive power minimum value Dh. The refractive power change graph GLPa is a graph showing changes in the last accommodation power Da during accommodation measurement. The fixation target conversion graph GLPb is a graph showing changes in presenting distance of fixation target during accommodation measurement. In the refractive power change graph GLPa and the fixation target conversion graph GLPb, the lateral axis indicates time (seconds) and the vertical axis indicates diopter (diopter value). The control part 70 plots the graphs GLPa and GLPb in the lateral axis corresponding to the elapsed time from the start of accommodation measurement. Specifically, the refractive power change graph GLPa and the fixation target conversion graph GLPb in the present embodiment are updated every time one cycle (S106 to S113) has passed since the start of measurement and thus the graph extends to the right from the start of measurement to the completion of measurement.

The refractive power change graph GLPa and the fixation target conversion graph GLPb are displayed in a superimposing manner on the anterior segment image F on the screen in the accommodation measuring mode for observing the anterior segment of the eye E. To prevent loss of visibility of the anterior segment image F, those graphs GLPa and GLPb are arranged in a right lower section of a display region of the monitor 7 by use of 20% or less of the entire display region of the monitor 7 with respect to the anterior segment image F displayed on the entire display region of the monitor 7. With such an arrangement, the graphs GLPa and GLPb are less likely to overlap not only a region showing the pupil of the eye E but also a region showing the iris displayed on the monitor 7. Accordingly, the examiner is allowed to check a progressing condition of the accommodation measurement (a tracking state of the refractive power of the eye E with respect to the presenting distance of the fixation target) while appropriately observing the anterior segment image F of the eye E. In the measurement in the present embodiment, the graphs GLPa and GLPb are updated (additional plotting) after a lapse of a predetermined time (e.g., one updating per two cycles) to correspond to the resolution of the monitor 7. The refractive power change graph GLPa changes in color in a vertical direction within a plotted region as will be mentioned later.

<Step S113>

The control part 70 stores in the memory 75 the eye refractive power of the eye E in each presenting position in association therewith. Herein, the memory 75 is also used as holding means (peak holding means) to hold a maximum value or a minimum value of the refractive power during measurement. If a refractive power exceeding the maximum value or the minimum value held (stored) in the memory 75 is obtained during measurement, the control part 70 updates the maximum value or the minimum value held at a predetermined address in the memory 75. As above, the control part 70 judges whether or not accommodation measurement should be terminated, and then terminates movement of the fixation target based on this judgment result. For instance, when it is determined that a predetermined condition is satisfied, i.e., if the elapsed time from the measurement start exceeds 30 seconds, if the maximum value of the refractive power during accommodation measurement remains unchanged for 6 seconds or more, or if the time for which the fixation target is stopped exceeds 6 seconds, the accommodation measurement is completed (S 113: YES). When the predetermined condition is not satisfied (S 113: NO), the flow goes to step S106 to continue the accommodation measurement.

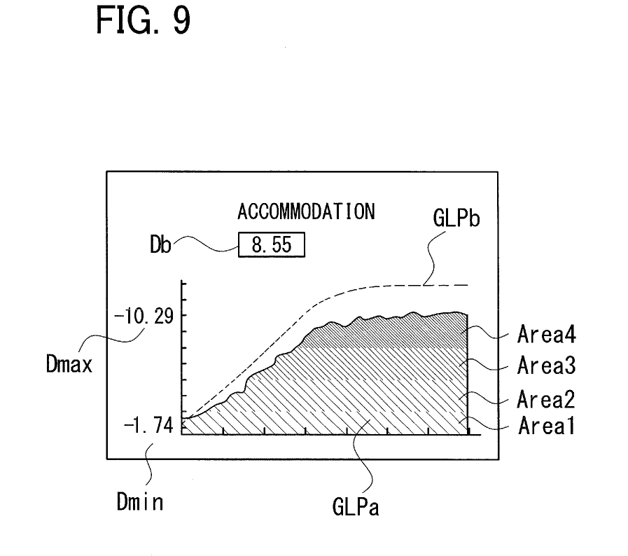

When the condition of measurement completion is satisfied, the monitor 7 is caused to display a pattern (icon) for shifting to a screen allowing the examiner to check a result of accommodation measurement. FIG. 9 illustrates a screen displayed when the examiner pushes an accommodation result display switch not shown of the switch part 8 to check the result of accommodation measurement. This accommodation result screen displays the measurement results including an accommodation power Db of the eye E, a near point value Dmax based on the maximum value of the refractive power measured during accommodation measurement, a far point value Dmin based on the minimum value of the refractive power measured during accommodation measurement, the refractive power change graph GLPa, and the fixation target conversion graph GLPb. The refractive power change graph GLPa is plotted with different colors in the vertical direction. A region Area 1 is indicated in light blue, a region Area 2 is indicated in green, a region Area 3 is indicated in yellow, and a region Area 4 is indicated in orange. An area near each boundary between the regions Area 1 to Area 4 is displayed in respective intermediate color. The graph extending in the vertical direction plotted in different colors in this manner enables the examiner to easily perceive changes of the tracking state of the examinee's eye E during accommodation measurement and also grasp the maximum accommodation power on the accommodation result screen.

In the present embodiment, the control part 70 calculates the accommodation power Db of the eye E based on the maximum value and the minimum value of the eye refractive power of the eye E in each presenting position when the presenting position of the fixation target is moved from the far distance to the near distance. This can enhance the property of measuring accommodation power.

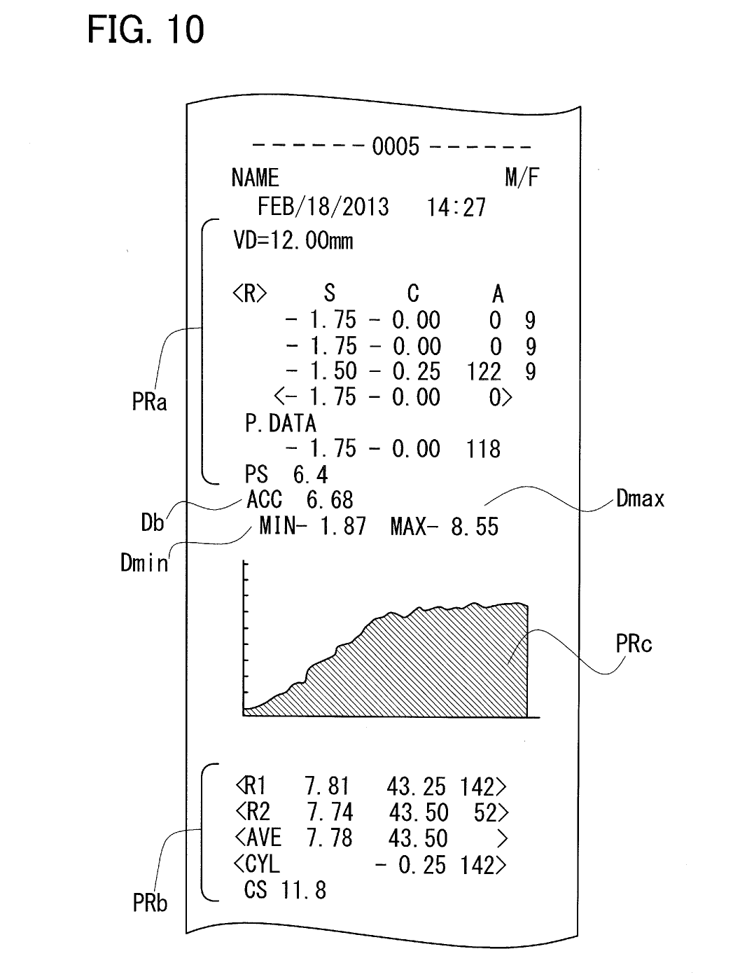

When the examiner operates a print switch not shown provided in the switch part 8 while the accommodation result screen is being displayed, the control part 70 controls a printer 78 to print the measurement result. FIG. 10 shows an example of a printed sheet output by a thermal printer. Measurement results printed on the printed sheet include information PRa and PRb measured in the objective far-vision refractive-power measuring mode, and further the accommodation power Db of the eye E measured in the accommodation measuring mode, the near point value Dmax based on the maximum value of the refractive power measured during accommodation measurement, the far point value Dmin based on the minimum value of the refractive power measured during accommodation measurement, and a refractive power change graph PRc for printing.

In the present embodiment, the start position of the accommodation measuring mode is determined and set based on the refractive power measured in the objective far-vision refractive-power measuring mode, but is not limited thereto. It may be arranged to measure the refractive power of an examinee's eye under the same conditions as those in the objective far-vision refractive-power measuring mode upon depression of the start switch and determine the far vision position, and move the fixation target to the presenting position.

The above explanation exemplifies the measurement optical system to obtain a ring pattern image formed by the fundus reflection light, but is not limited thereto. The present disclosure is also applicable to any apparatus arranged to move the presenting distance of a fixation target and measure accommodation power of an examinee's eye E based on objective measurement of refractive power of the eye E. For instance, a measurement optical system may be arranged to project a spot index onto a fundus Ef of the examinee's eye E to obtain wavefront aberration of the eye E and detect fundus reflection light using a Shack-Hartmann sensor.

In the above description, when the control amount is to be changed based on a monitoring result, the control amount is changed if the presenting position information of the fixation target and the measurement results of the eye refractive power at the presenting distance do not satisfy the first allowable range. The present disclosure is however not limited thereto.

For instance, the control part 70 has only to determine the tracking state of the examinee's eye with respect to movement of the fixation target (e.g., whether or not the tracking state is good) based on changes in the measurement results of the eye refractive power while the presenting position of the fixation target is moved from the far distance to the near distance. In this case, when it is determined that the tracking state becomes deteriorated, the control part 70 changes the control amount.

In more detail, the control part 70 may change the control amount according to the measurement results of the eye refractive power at the presenting distance. Specifically, for example, when an amount of change in eye refractive power per unit of time is decreased, it is conceived that the tracking ability of the examinee's eye with respect to the fixation target has changed. Therefore, the control part 70 may change the control amount (e.g., the moving speed of the fixation target or the moving amount of the fixation target at each step) according to the change amount of the eye refractive power per unit of time. This allows the examinee to follow the fixation target. Accordingly, an actual accommodation power can be smoothly measured. The control amount is required only to increase when the change amount is increased and to decrease when the change amount is decreased (or when the change amount becomes zero).

The control part 70 may also change the control amount according to for example the presenting position of the fixation target. For instance, as the presenting distance of the fixation target comes closer to the examinee's eye E, a larger accommodation strain is put on the examinee, making it difficult for the examinee's eye E to track or follow the fixation target. Therefore, it may be arranged to change the control amount (e.g., the moving speed of the fixation target or the moving amount of the fixation target at each step) according to the presenting position of the fixation target. This allows the examinee to track or follow the fixation target. Accordingly, an actual accommodation power can be smoothly measured. The control amount is required only to increase when the presenting distance is far from the examinee's eye and to decrease when the presenting distance is close to the examinee's eye.

In the above explanation, when the presenting position of the fixation target and the measurement results of the eye refractive power at the presenting distance do not satisfy the allowable range, the moving direction of the fixation target is changed or the movement of the fixation target is temporarily stopped. However, the present disclosure is not limited thereto. Specifically, the control part 70 determines whether or not the examinee's eye is able to track the movement of the fixation target based on changes in the measurement results of the eye refractive power while the presenting position of the fixation target is moved from the far distance to the near distance. When it is determined that the examinee's eye is unable to track the fixation target, the control part 70 has only to change the moving direction of the fixation target or temporarily stop the movement of the fixation target. For instance, the control part 70 determines whether or not the examinee's eye is tracking the fixation target according to whether or not the change amount of eye refractive power per unit of time satisfies the allowable range. When the control part 70 determines that the change amount does not satisfy the allowable range, the control part 70 has only to change the moving direction of the fixation target or temporarily stop the movement of the fixation target.

In the above explanation, the movement of the fixation target is stopped after a lapse of a predetermined time from when the change in measurement result of eye refractive power is turned into a decline. However, the present disclosure is not limited thereto. Specifically, the control part 70 determines whether or not the examinee's eye is able to track the movement of the fixation target based on the changes in measurement results of eye refractive power while the presenting position of the fixation target is moved from the far distance to the near distance. After a certain amount of time for which the examinee's eye is unable to track the fixation target, the control part 70 may stop the movement of the fixation target. For instance, even though the control amount is changed, the control part 70 determines whether or not the examinee's eye is able to track the fixation target according to whether or not the change amount of eye refractive power per unit of time satisfies the allowable range. The control part 70 may stop the movement of the fixation target after the certain amount of time for which the change amount does not satisfy the allowable range.

The method of changing the control amount in the above explanation exemplifies the case where the accommodation power of the examinee's eye based on the eye refractive power obtained in each presenting position while the presenting position of the fixation target is moved from the far distance to the near distance. The disclosure is however not limited thereto. For instance, the technique of the present embodiment is also applicable to the case where the fixation target is moved from the far distance to the near distance when the eye refractive power of the examinee's eye in the near distance is to be measured.

Reference signs list

| | | | |
|---|---|---|---|
| 4 | Measuring part | 6 | XYZ drive part |
| 7 | Monitor | 8 | Switch part |
| 10 | Measuring optical system | 22 | Imaging device |
| 30 | Fixation target presenting | 32 | Fixation target plate |
| | optical system | | |
| 37 | Drive part | 50 | Observation optical system |
| 52 | Imaging device | 70 | Control part |
| 75 | Memory | 77 | Image processing part |

What is claimed is:

1. An eye refractive power measuring apparatus including:
a measuring part configured to project measurement light onto a fundus of an examinee's eye and measure eye refractive power of the eye based on reflection light of the measurement light from the fundus;
a fixation target presenting part configured to present a fixation target to the eye;
a drive part configured to move a presenting position of the fixation target to be presented to the eye; and
a control part configured to control the drive part to move the presenting position of the fixation target from far distance to near distance,
the apparatus being configured to measure the eye refractive power in at least a far position and a near position,
wherein the control part controls the drive part to change a control amount of the drive part based on a change in measurement results of the eye refractive power while the fixation target is moved from the far distance to the near distance.

2. The eye refractive power measuring apparatus according to claim 1, wherein, when the control amount is to be changed, the control part changes at least one of a moving speed of the fixation target while the fixation target is moved from the far distance to the near distance and a moving amount at each step while the fixation target is moved on a step-by-step basis from the far distance to the near distance.

3. The eye refractive power measuring apparatus according to claim 1, wherein the measuring part is configured to measure accommodation power of the examinee's eye based on an eye refractive power of the examinee's eye in each presenting position while the presenting position of the fixation target is moved from the far distance to the near distance.

4. The eye refractive power measuring apparatus according to claim 1, wherein the control part is configured to change a moving direction of the fixation target or temporarily stop movement of the fixation target based on the change in measurement results of the eye refractive power while the presenting position of the fixation target is moved from the far distance to the near distance.

5. The eye refractive power measuring apparatus according to claim 1, wherein the control part is configured to determine whether or not the examinee's eye is able to track movement of the fixation target based on the change in measurement results of the eye refractive power while the presenting position of the fixation target is moved from the far distance to the near distance, and terminate the movement of the fixation target after a predetermined period of time for which the eye is unable to track the fixation target.

6. The eye refractive power measuring apparatus according to claim 1, wherein, when the accommodation power of the examinee's eye is to be measured by the measuring part, the control part changes a method of measuring the eye refractive power between first refractive power measurement to measure the eye refractive power to determine an initial presenting position of the fixation target and second refractive power measurement to measure the eye refractive power by moving the presenting position.

7. The eye refractive power measuring apparatus according to claim 1, wherein the measuring part includes a two-dimensional imaging device arranged to store the reflection light, and the measuring part is arranged to obtain a refractive power by use of an output signal from the two-dimensional imaging device while the fixation target remains stationary.
